(19) United States
(12) Patent Application Publication
(10) Pub. No.: US 2013/0312793 A1
(43) Pub. Date: Nov. 28, 2013

(54) METHOD AND DEVICE FOR CLEANING MEDICAL INSTRUMENTS

(71) Applicant: Georgios Ionidis, Zurich (CH)

(72) Inventor: Georgios Ionidis, Zurich (CH)

(73) Assignee: ORO CLEAN CHEMIE AG, Fehraltorf (CH)

(21) Appl. No.: 13/984,768

(22) PCT Filed: Feb. 6, 2012
(86) PCT No.: PCT/EP2012/000517

§ 371 Date: Aug. 9, 2013

(30) Foreign Application Priority Data
Feb. 11, 2011 (EP) 11001127.7

(51) Int. Cl. A61B 19/00

(52) U.S. Cl. CPC A61B 19/34
USPC 134/22.19; 134/94.1; 134/22.1

(57) ABSTRACT

The device according to the invention is used to clean medical instruments having at least one instrument cavity and at least two openings, wherein the device has a first fastening module for detachably fastening the medical instrument and a second fastening module for detachably fastening a first container for receiving liquid (insert in drawing) and/or air (insert in drawing), wherein the device comprises a cleaning device cavity which is designed such that when the medical instrument and container are fastened, the liquid and/or air can be flushed from the interior of the container through the cleaning device cavity into the instrument cavity. According to the invention, the device has a first and a second operating position, wherein, in the first operating position, the liquid can be flushed out of the container through the cleaning device cavity into the instrument cavity and, in the second operating position, air can be flushed out of the environment through the container and the cleaning device cavity into the instrument cavity.

The invention relates to a device and a method for cleaning and/or disinfecting and/or caring for medical, in particular dental, instruments with a cavity, especially dental preparation instruments with drive elements for rotating instruments, like for example dental hand-pieces, elbows and/or nozzles. In dentistry there is extensive use of preparation instruments with which teeth are treated using rotating drill, grinder, and polishing devices. During this usage blood, saliva, water, grinding bodies, and/or polishing bodies penetrate into the interior of the medical instrument and contaminate it. During further usage these contaminants can come in contact with the patient and result in infections in the patient. These infections should be avoided. The suctioned impurities also result in deposits and bio-film growth, which damages precision parts by abrasion or jamming and reduce the life-expectancy of the medical instruments. Another problem with the use of the instruments lies in the fact that the instruments feature water-conducting systems. Calcium deposits can occur in these water-conducting systems, which block the water-conducting system. For these reasons the interior of the medical instruments must be cleaned and disinfected at regular intervals.

Known from DE 43 26 956 is a method for cleaning dental instruments with a cavity. In this method the instrument to be cleaned is placed in a housing featuring at least two openings. The housing is thereby connected via one opening to the vacuum network of the dental practice. Ambient air is suctioned in through a second opening. The instrument to be cleaned is so placed in the housing that during the cleaning process the ambient air travels with the cleaning liquid in the housing and this flows through the cavity of the dental instrument into the vacuum network.

The instrument is cleaned in this manner. The cleaning effect can also be enhanced by a rotation of the instrument. This method has the disadvantage that during the cleaning a mixture of air and cleaning solution is brought into the cavity. There is no control over whether sufficient cleaning agent was brought into the cavity. Another disadvantage consists of the cleaning and disinfecting agent being able to exit in an uncontrolled manner through the second opening and thus the dental practice can be contaminated. Another disadvantage consists of the cleaning agent removing the lubricating agent that is present. For that reason, the instrument must be oiled after the cleaning. The container must be cleaned and filled with oil in a second step in the method introduced in DE 43 26 956. Finally the container is again connected to the vacuum network and an air-oil mixture is brought into the instrument. This method is complicated. Furthermore the acquisition and maintenance of such devices is very expensive.

The object of the invention is therefore to make available a cleaning device and method to clean medical instruments, which guarantees a reliable cleaning, disinfection and maintenance of the medical instrument, is simple to use, and is convenient to manufacture and use.

This object is achieved by a cleaning device and method to clean medical instruments, which exhibits the features of the independent patent claim. Other advantageous embodiments can be extracted from the dependent claims.

The inventive cleaning device for the accommodation of medical instruments for cleaning has at least one instrument cavity and at least two openings. The cleaning instrument thereby exhibits a first fastening module for detachably fastening the medical instrument and a second fastening module for detachably fastening a first container for receiving liquid and/or air, which is so configured that with a fastened medical instrument and a fastened container, the liquid and/or the air can be flushed from the interior of the container through the cleaning device cavity into the instrument cavity.

The cleaning device features a first and second operating position wherein, in the first operating position, the liquid can be flushed out of the container through the cleaning device cavity into the instrument cavity, and in the second operating position ambient air is flushed through the container and the cleaning device cavity into the instrument cavity.

The inventive cleaning device is especially suited for dental instruments, like dental hand-pieces, elbow pieces, or nozzles. The cleaning device, however, can also be used for the cleaning of other medical or veterinary instruments, like for example endoscopes, hollow needles, or other instruments with cavities.

As a rule, the instrument cavity features exactly two openings. During the cleaning procedure the cleaning liquid is flushed through one opening into the instrument cavity and the cleaning liquid exits from the second opening out of the instrument cavity. In this manner the instrument cavity is rinsed with cleaning liquid.

Usually the medical instrument features exactly one instrument cavity, which must be cleaned. Many instruments, however, exhibit more than one instrument cavity. There are, for example, instruments with additional air and/or water canals, which run parallel to the first instrument cavity. It is also possible that the instrument features a canal to emplace a probe. Many of the canals end in jets, which transport a liquid flowing in a canal to a certain location. For example, dental hand-pieces, elbow piece, or nozzles are often so constructed that water can be sprayed from interior canals through a jet on to a tooth, in order to cool it during drilling. These additional instrument cavities, also named canals, are also affected by contaminants and can likewise be cleaned with the inventive cleaning device. To do so, the additional instrument cavities must also be able to be connected to the first fastening module with the cleaning device cavity. During the cleaning procedure the instrument cavities are simultaneously rinsed with the liquid, which previously was located in the fastened container.

The cleaning device is preferably so constructed that no gaps and/or dead spaces are located in the interior in which air can remain in these gaps or dead spaces during the flushing with air in the second operating position. When flushing with air, such a construction prevents any small quantities of liquid entering into the medical instrument from the gaps and/or dead spaces from extending the duration of emptying.

The cleaning device is preferably so constructed that it is possible to suction ambient air into the container to compensate vacuum pressure during the dispersion of liquid or air in the first as well as the second operating position; the outflow of liquid from the container is thereby prevented in the first position. This can be achieved in that the air supply canals are narrow enough to prevent the flow of liquid because of surface tension and/or affinity of the material of the air supply canals. Advantageous is an air supply canal with at least one elbow or bend, because the elbow or bend also makes the outflow difficult. It is also possible by suitable adaptation of the length of the air supply canals and the number of elbows to prevent any exit of the liquid, wherein air can always flow through. The opening of the air supply canals to the atmosphere can be covered by a sterile filter. The sterile filter should exhibit a pore width of less than 0.2 mm in order to prevent penetration of bacteria.

Various receptacle devices for liquids can be used as the container. For example, rigid containers of metal, glass or plastic can be used. In principle, only rigid or slightly deformable containers should be used which during use do not collapse in order to create the necessary partial vacuum for air intake. Other designs of containers, however, are possible. Containers with an additional, closeable opening, for example, can be used through which the liquid can be refilled. It is also basically possible to permanently or temporarily connect these containers to a reservoir using a tube in order to replenish the solution.

Ideally the container is affixed to the fastening module with the help of a reversible, removable, threaded insert into which another part can simply be inserted, for example, with another threaded diameter. This offers the advantage that various containers can be used on the cleaning device or can be modified in a simple and quick manner.

It is possible that a second container can be affixed to the second fastening module and the second receptacle element features a selection module, which enables the selective removal of liquid from the first or the second container. Thus one container can contain a cleaning substance and a second container a disinfecting liquid. A selection can be made by movement of a rotatable disc as to which liquid should be pumped through the dosing pump. The air blow-out step remains the same in this embodiment.

Ideally a liquid is used whose density is greater than the density of the atmospheric air. The operating positions of the cleaning instrument are so selected that in the first operating position the liquid is located before the second fastening module and in the second operating position the atmospheric air is located before the second fastening module. The device preferably features a dosing pump wherein by means of this dosing pump liquid can be flushed in the first operating position from the container through the cleaning device cavity into the instrument cavity and by means of the dosing pump ambient air is flushed in the second operating position through the container and the cleaning device cavity into the instrument cavity. The dosing pump can have a fixed volume, which constantly releases an equal quantity of liquid. It is alternatively conceivable to use a dosing pump with adjustable volume (e.g. by limiting the piston stroke) whose release volume can be varied.

Ideally there can be a change between the first and second operating position by swiveling the cleaning device by approximately 180° in the direction of gravity. The liquid can thereby be flushed in the first operating position through the reception module because of gravity. The liquid is drawn under the influence of the dosing pump and thereby flows from the container through the cleaning device through the instrument cavity and exits from an opening of the instrument cavity. All cavities are filled with liquid in this manner. After sufficient liquid is flushed through the instrument cavity, the cleaning device is swiveled by 180° in the direction of gravity and assumes the second operating position. In this second operating position air is drawn from the container under the influence of the dosing pump through the cleaning device and the instrument cavity and exits from an opening of the instrument cavity. The air is thereby flushed through the instrument cavity. This air travels with the liquid located in the instrument cavity and in this manner removes liquid from the instrument cavity. Ambient air is suctioned into the container for compensation of vacuum pressure during the dispersion of liquid or air. The inventive method to clean a medical instrument uses a previously described inventive cleaning device.

In the inventive method the medical instrument is fastened in the cleaning device by means of a fastening module. A receptacle element is affixed on the cleaning device by means of a second fastening module. Located in the receptacle element is a liquid by means of which the instrument cavity is cleaned. The cleaning device exhibits a cleaning device cavity, which is so designed that with a fastened medical instrument and a fastened container, the liquid and/or the air is flushed from the interior of the container through the cleaning instrument cavity into the instrument cavity. The cleaning device thereby features a first and a second operating position. In the first operating position the liquid is flushed out of the container through the cleaning device cavity into the instrument cavity and in the second operating position ambient air is flushed through the container and the cleaning device cavity into the instrument cavity. In this manner the instrument cavity is cleaned in a first step by means of the flowing liquid and in a second step the instrument cavity is dried by means of the air flowing through it.

Since this method brings pure liquid into the instrument and not an air/liquid mixture, a complete moistening of all surfaces is guaranteed.

The cleaning and follow-on air rinsing occurs in this manner without coupling of the medical instrument to the cleaning device, which simplifies the work.

These methods are advantageous since they do not require pressurized gas for rinsing the medical instruments with liquid or gas. As a result no expensive connections to gas lines are necessary. By the non-use of pressurized gas, the formation of spray and in particular small drops of cleaning liquids or deposits in the medical instrument are avoided. This represents a special advantage, since the inhalation of such sprays or drops can result in poisoning, irritations or infections in cleaning personnel. Furthermore the use of pressurized gas has the additional disadvantage that a complete moistening of all surfaces cannot be guaranteed.

An additional advantage of the method is the possibility of filling the medical instrument with liquid without a partial vacuum or vacuum having to be applied. As a result the expensive and technically complicated creation of a vacuum is not necessary. An additional disadvantage in using a vacuum is that a complete moistening of all surfaces cannot be guaranteed with certainty. Here, too, there appears the problem of droplet formation and its associated disadvantages.

Another advantage of the method consists of no significant increase of the pressure occurring in the interior of the container. As a result, no special measures are necessary, which would be required for pressurized components. Pressurized components also involve the danger that they may burst and distribute liquid and gas in the area, which represent a hazard to the cleaning personnel.

Ideally the fastening to the first fastening module occurs with the help of an adapter. The use of adapters enables the fastening of various dental and medical instruments with different opening configurations onto the cleaning device. It is also advantageous to design the adapter as simply as possible and provide for a one-time use. The one-time use of the adapter embodies the advantage that the adapter is replaced after each use and thus hygienic certainty can be significantly increased in a simple manner.

Ideally the fastening of the adapter in the cleaning device occurs via a standard connection, like for example a standardized Luer lock connection. This also enables the fastening of other devices between the adapter and the cleaning device, assuming these other devices also have available a standardized connection, like for example a Luer lock connection. One possible connection, for example, is a filter, which is fastened between the adapter and the cleaning device, wherein the filter serves to clean the liquid before entry into the instrument cavity. Naturally other standardized connections can also be used.

The adapter can be connected directly onto the cleaning device. An alternative connection of the adapter to the cleaning device can be via a tube.

If the medical or dental instruments feature moving or rotating parts, it is advantageous if such parts can be activated or rotated during the cleaning, disinfecting and/or maintenance, since there is the assurance because of this activation or rotation that all surfaces come in contact with the liquid. It is not necessary that this activation be performed in such a manner that the full function of the medical or dental instrument is achieved during the cleaning. It suffices when the moving parts run through all movement and rotation cycles several times. It is sufficient, for example, if the moving parts of a hand-piece are rotated several times under power. This rotation can occur manually or by motor drive.

The liquid used is ideally a cleaning liquid, which preferably contains a disinfectant. This disinfectant is so selected that it disinfects the instrument cavity during cleaning. A disinfectant effective against viruses is defined when the disinfectant is able, for example within a defined exposure time, to reduce the virus titer of the test virus strains (corresponding to the quantity of infectious viruses present in a cell culture per volume unit) by at least 4 decimal-logarithmic levels or 4 log-levels. A reduction by one log-level corresponds to a tenfold or 90% reduction of the virus strains. Accordingly a reduction by 4 log-levels corresponds to a 10⁴-fold or 99.99% reduction of virus strains. If for example 10⁶-fold virus strains are initially present, only 10² virus strains survive a reduction by 4 log-levels.

Therefore so-called virucidal disinfectants are used which are effective against both enveloped viruses as well as non-enveloped viruses. In general with alcohol disinfectants that includes an effectiveness against non-enveloped viruses as well as an effectiveness against all enveloped viruses. According to Standard EN 14476 (from 2005) a disinfectant is virucidal when it is able to inactivate the following non-enveloped virus strains under defined test conditions:
Polio virus type 1, LSc-2ab,
Adeno virus type 5, adenoid 75 strain, ATCC VR-5
   The Guideline of the German Association to Combat Viral Illness (DW) Inc. and the Robert Koch Institute (RKI) in the version of 2005 (DVV/RKI Guideline) requires an expanded test procedure for virucidal disinfectants in, which additional enveloped virus strains are included:
Vaccine virus, Elstree strain
Polio virus type 1, LSc-2ab—Adeno virus type 5, Adenoid strain 75
Polyoma virus (SV 40), Strain 777

During cleaning the medical instrument is not used on the patient. The use of a cleaning liquid, which acts in an irritating or corrosive manner on living tissue, is therefore possible and reasonable, assuming that this liquid is completely removed from the medical instrument at the end of the cleaning.

A liquid is preferably used for cleaning, which features several components. These components can exhibit a homogenous mixture. It can be useful, however, to use a non-homogenous mixture.

Ideally one component is oil. This embodiment is especially advantageous, since during the cleaning process oil is already brought into the instrument cavity in the cleaning process. A part of the oil remains in the instrument cavity after the cleaning process. The instrument cavity is already lubricated in this manner during the cleaning process. The additional step of a follow-on lubrication of the cleaning cavity is omitted and as a result simplifies the cleaning process. In addition the user does not need to store additional liquid for lubricating. That also represents an advantage. Especially advantageous is the presence of oil in cleaning liquids containing alcohol, since oil-free liquids containing alcohol completely remove the necessary oil present on the medical instrument. If after a cleaning with an oil-free, alcohol-based liquid the follow-on lubrication is inadvertently forgotten, major damage can occur with rotating parts, like for example dental turbines, which operate at up to 500,000 rpm. The combination of the inventive cleaning device with an oil-based liquid thus represents a basic advantage for the user.

The volume portion of the oil in the liquid is ideally greater than 0.2% and less than 10%. A volume portion of 0.3% to 0.7% is especially advantageous. The use of high performance dental oil has shown especially good results.

Ideally one of the components consists of short-chain alcohols and the volume portion of the short-chain alcohols in the liquid is greater than 40% and less than 80%. Especially good cleaning results have been shown for a volume portion of short-chain alcohols in the liquid greater than 60% and less than 75%. Solutions with such a portion of short-chain alcohols have low corrosiveness for iron.

The liquid ideally contains inorganic acids since such acids increase the anti-microbial effectiveness and remove calcium deposits in water-conducting canals. Examples of such acids are, among others, hydrochloric acid, sulfuric acid, amidosulfonic acid, and phosphoric acid.

Ideally the liquid contains phosphoric acid and the volume portion of the phosphoric acid in the liquid is greater than 0.1% and less than 2%. Especially good results have been seen for a volume portion of the phosphoric acid in the liquid greater than 0.7% and less than 1%. Phosphoric acid exhibits additional anti-corrosive characteristics.

Ideally the liquid used is a disinfectant, which is effective against all viruses, all bacteria, and all Candida.

Other cleaning liquids are however possible. Thus, for example, a cleaning solution on the basis of water and surfactants can be used. Such cleaning liquids can also contain additional components, which provide an additional advantage. Some possible additional components are, for example, enzymes, which support protein removal. Also advantageous is the use of the previously known and recognized cleaning liquids, which are tested for example according to International Standard 15883. Also possible are disinfectants , which are based on a diluted solution of biocides, such as for example alcohols, quaternary ammonium compounds, alkylamine derivates, guanidine derivates, aldehydes, halogens, peroxide compounds, phenols and others. Also possible are maintenance liquids for regular use, such as for example lubricants, which are based on a homogeneous mixture of short-chain alcohols and oils or a stable suspension of oil in aqueous liquid. Also possible are maintenance liquids for one-time or occasional use, such as a solution of fluro-surfactants for a one-time coating of the interior surfaces of the medical instrument. These liquids can be made available to the user, for example, as ready-to-use solutions or concentrates for dilution with water. It is also possible to make available to the user a multi-component set to produce the liquid before use.

Ideally the cleaning, disinfecting or maintenance liquid attains its effectiveness in a short time. The effectiveness is preferably attained within a minute. With longer effective times there is the danger that the cleaning procedure will cease before the start of the effectiveness. Furthermore with longer required times to attain effectiveness, the danger increases that the materials of the medical instruments will be attacked by the cleaning solution.

Ideally the cleaning, disinfecting or maintenance liquid contains no components, which are toxic for humans and act corrosively on medical instruments in a concentration, which exceeds 0.1%. Such undesirable components are, for example, salts of heavy metals, like mercury compounds.

Ideally a liquid is used for cleaning, disinfecting, or maintenance, which is suitable for both interior and exterior use on a medical instrument.

An advantage of the method is that the cleaning, disinfecting or maintenance can be performed at room temperature. Understood as room temperature is a temperature from 15-25° C. The method, however, can also be performed without problems at temperatures from 5-40° C.

Methods are known from the prior art, which basically require higher temperatures. Usually these methods are performed at temperatures from 60-135° C. These higher temperatures lead to a series of problems. For instance, the cleaned instruments will be stressed more severely, which promotes fatigue cracks and/or fatigue corrosion, which shortens the life expectancy of expensive medical and dental instruments. In addition, thermal methods require expensive devices, like autoclaves for example. Furthermore thermal methods are time-intense, since a longer waiting time is required for cooling of the instrument.

The inventive device is described below using embodiments as schematically depicted in the figures. Shown are:

FIG. 1: A cross-section through a first embodiment of the inventive cleaning device with a fastened container and medical instrument in the second operating position;

FIG. 2: A cross-section through a second embodiment of the inventive cleaning device with a fastened container and medical instrument in the second operating position;

FIG. 3a: A cross-section through the first embodiment of the inventive cleaning device with a fastened container in the first operating position;

FIG. 3b: A cross-section through the first embodiment of the inventive cleaning device with a fastened container in the first operating position during the pumping procedure;

FIG. 3c: A cross-section through the first embodiment of the inventive cleaning device with a fastened container in the first operating position;

FIG. 3d: A cross-section through the first embodiment of the inventive cleaning device with a fastened container in the first operating position in the refill mode;

### DESCRIPTION OF THE FIGURES

FIG. 1 shows a cross-section through a first embodiment of the inventive cleaning device 100 with a fastened container 7 and medical instrument 60 in the second operating position. The container 7 serves to accommodate liquids 80 to clean the medical instrument 60. The cleaning device 100 is so designed that liquid or gas can be brought from the interior of the container through the cleaning device into the instrument cavity 62 of the medical instrument 60. The cleaning device 100 exhibits a second fastening module 6 to detachably fasten the container 7. The second fastening module 6 features a threaded insert 75, which is reversibly removable from the cleaning device 100. This reversibly removable threaded insert is cylindrically shaped and exhibits a threading 49 on the interior side. One end of this part is open and serves to accommodate the container 7. The other end of this part is closed except for opening 58 for passage of liquid from the container 7 into the cleaning device 100, and except for an outlet opening 50 for ambient air of the air intake system into the container 7. On the exterior side it exhibits devices for reversible removal and fastening in the fastening module 6. This device for reversible removal and fastening is in the embodiment depicted here an interlocking retention groove 10 and a locking protrusion 56. Other embodiments of the reversible fastening and removal are, however, possible. For example, an interacting screw connection or a groove and spring connection can be used. The reversibly removable part of the fastening module 6 has the advantage that another part with another threaded diameter can be simply used. This offers the advantage that the cleaning device 100 can be used for various containers and can be simply and quickly converted. In order to ensure that the reversibly removable part of the fastening module 6 is correctly positioned, an interacting positioning projection 9 and a positioning indentation 47 are positioned on this part and the cleaning device. The container 7 in this embodiment is a cylinder with a formed cylinder thread 48. The cylinder threading 48 interacts with a threading 49 of the threaded insert 75 of the second fastening module 6. The axis of the threading 49 of the threaded insert 75 and the cylinder threading 48 are aligned vertically both in the first as well as the second operating position. If the liquid flows out of the container 7 during the cleaning process, a partial vacuum is the result. In order to allow air into the container 7 and compensate for the partial vacuum, the second fastening module 6 features an air canal 52 by means of which ambient air can be brought into the interior of the container 7. The air canal 52 must thereby be so designed that the ambient air can indeed move into the container 7. The liquid, however, cannot exit from the container 7 through the air canal 52 into the environment. In order to solve this problem the air canal 52 is so designed that in the second operating position the air canal 52 has its inlet opening 51 on the bottom side of the second fastening module 6. The air canal 52 is operated the majority of the time in the vertical direction in the fastening module 6. In order to impede the liquid exit, a space 54 is positioned on the air canal in which any possibly exiting liquid is collected. Furthermore the air canal 52 features a horizontal canal section 53. The combination of a horizontal and vertical canal section form a type of labyrinth, which impedes the exit of the liquid into the surroundings.

An inlet valve is positioned within the cleaning device 100 at the opening to the liquid outlet 58 between the container 7 and the cleaning device 100 on the inside. The inlet valve is positioned in an inlet valve cylinder 17, which extends in the second operating position in a vertical direction above the opening 58. The inlet valve features an inlet valve ball 55 and an inlet valve spring 12. The inlet valve spring 12 is under tension. The inlet valve spring 12 exhibits two ends wherein the one end rests on a support plate 11a, 11b and the other end on the inlet valve ball 55. The inlet valve ball 55 is so designed that it is held on a support platform 57 positioned around the opening 58 because of the spring force of the inlet spring 12. The inlet valve cylinder 17 exhibits a vertically extending inlet opening 14 into a cylinder 2. Liquid flows through this inlet opening 14 from the inlet valve cylinder 17 into the cylinder 2. The support platform 57 is molded on the reversibly removable part of the fastening module 6. In order to prevent liquid from exiting at the opening 58 a sealing ring 46 is positioned around the support platform 57. The cleaning device 100 features a cylinder housing 1. The axis of the cylinder housing 1 is aligned horizontal to the force of gravity both in the first as well as the second operating position. By rotating the cleaning device 100 by approximately 180° around the axis of the cylinder housing 1 a change is made between the first and second operating position. A cylinder 2 is placed inside the cylinder housing 1. The axis of the cylinder 2 is aligned horizontal to the force of gravity in the second operating position. The cylinder 2 is affixed in the cylinder housing 1. In this embodiment the cylinder 2 features a locking protrusion 24, which interacts with a locking indentation 8 molded on the cylinder housing 1. The cylinder 2 features two opposing end walls. The first end wall exhibits a piston opening 27. A hollow piston 4 is mounted in this piston opening 27 so as to move horizontally. The hollow piston 4 features a first and second end. Molded on the first end of the hollow piston 4 is a hollow piston head 22. This hollow piston head 22 is placed in the interior of the cylinder 2, the so-called cylinder space 13. The hollow cylinder head 22 is so designed that it interacts with the interior wall 20 of the cylinder 2 such that a minimum of liquid exits between the hollow piston head 22 and the interior wall 20. In order to further impede the passage of liquid a seal 21 is placed between the hollow piston head 22 and the interior wall 20. The second end of the hollow piston 4 is connected to the instrument cavity 62 of the medical instrument 60 via an adapter 5. A hollow piston spring 23 is positioned in the interior of the hollow piston 4, which is tensioned in the horizontal direction. The hollow piston spring 23 features a first and a second end. The first end rests on a first support platform 15 molded on the cylinder housing 1. The second end rests on a second support platform 26 of the hollow piston spring 23 positioned in the hollow piston interior space 25. The second support platform 26 separates a discharge valve area 31 from the hollow piston interior space 25. The second support platform 26 features a discharge valve opening 29 through which liquid can flow. A discharge valve is placed in the discharge valve area 31, which exhibits a discharge filter spring 32 and a discharge valve ball 28. The discharge valve ball 28 is so positioned in front of the discharge valve opening 29 that any uncontrolled discharge of liquid is prevented. An additional support platform 45 for the discharge valve ball 28 is molded on the support platform 26 to position the discharge valve ball 28. The discharge valve spring 32 is tensioned in the horizontal direction and features a first and a second end. The first end of the discharge valve spring 32 is positioned at the discharge valve ball 28. The second end of the discharge valve spring 32 is supported on a discharge valve spring support plate 44. The discharge valve spring support plate 44 can be realized in various ways. It is possible, for example, to mold it directly in the hollow piston 4. On the other hand, in this embodiment a ‘piston grip 3 is positioned between the hollow piston 4 and the adapter 5. An anatomically shaped curvature 33 is molded on the piston grip 3. By using the piston grip 3 the hollow piston 4 can be horizontally displaced. This horizontal displacement aids the transport of liquid and gas into the cleaning device 100. An anatomically shaped curvature 18 can likewise be molded on the cylinder housing 1. The anatomically shaped curvatures 33, 18 facilitate comfortable manipulation of the cleaning device 100. The discharge valve spring support plate 44 is molded on the piston grip 3. The hollow piston 4, the piston grip 3, and the adapter 5 are rigidly connected to each other. For that reason locking projections 30 are molded on the hollow piston 4, which interact with locking indentations 34 of the piston grip 3, and locking projections 41 are molded on the piston grip 3, which interact with the locking indentations 42 of the adapter 5. It would also be conceivable to shape the hollow piston 4, the piston grip 3, and the adapter 5 from a single piece. There is also the possibility of shaping the piston grip 3 and the adapter 5 from one piece. It would likewise be conceivable to shape the second fastening module 6 as an integral part of the cylinder housing 1.

The discharge valve spring support plate 44 features a discharge opening 36 through which liquid can flow. This discharge opening 36 is so constructed that it forms a jet 37. The jet 37 features a narrowing cross-section, which has the task of concentrating the liquid beam so that the liquid can be flushed into the medical instrument 60 with a greater velocity. The greater velocity has a positive effect on the cleaning behavior of the cleaning device 100. The adapter 5 facilitates the connection between the cleaning device 100 and the medical instrument 60. The adapter 5 is designed for the cleaning of a particular medical instrument 60. If another medical instrument 60 is to be cleaned with the cleaning device 100, only the adapter 5 need be changed. It is therefore advantageous to connect the adapter 5 with the remaining parts of the cleaning device 100 by means of a plug connection. In the embodiment shown, the adapter 5 is connected to the piston grip 3 by means of a matched joint. If the adapter 5 is to be changed, it can be simply removed from the piston grip 3 by a pulling motion. Another adapter can then be affixed on the piston grip 3 with a simple pressing motion. Immediately thereafter the cleaning device can be used for the cleaning of another medical instrument. The medical instrument 60 shown in FIG. 3 exhibits a tool head 61. Arranged in medical instrument 60 there is an instrument cavity 62, the so-called main canal. In addition, another instrument cavity, a so-called secondary canal 64, is positioned in the medical instrument 60. The main canal 62 features an intake opening 63 and a discharge opening 66. With a fastened cleaning instrument 100 the intake opening 63 of the main canal 62 is located in the extension of the axis of the hollow piston 4. In this design the water beam exiting from the jet 37 impacts directly on the intake opening 63 , which guarantees an optimal cleaning of the main canal 62. The secondary canal 64 exhibits an intake opening 65 and a discharge opening 67. The intake opening 65 is positioned in an axial direction and with a fastened cleaning device 100 is separated from the extension of the axis of the hollow piston 4. A cylinder-shaped adapter 5 with open ends is used for cleaning of this configuration of the medical instrument 60. The axis of the adapter 5 agrees with the axis of the hollow piston 4. The one end of the cylinder is connected to the piston grip 3 with the help of a matched joint 41, 42; the other end of the cylinder terminates in a form-lock on the medical instrument 60. The adapter 5 features a side opening 40 in the cylinder to clean the secondary canal 64. To prevent the discharge of liquid a first seal ring 38 is positioned in front of the side opening 40 and a second seal ring 39 after the side opening 40. The space between the first and second seal ring is designated by reference number 68 as the interior space between the first and second seal. The area inside the adapter 5 is designated as the adapter space 43.

The intake valve 12, 17, 55, the discharge valve 31, 32, 55, and the piston area 4 form the dosing pump 79.

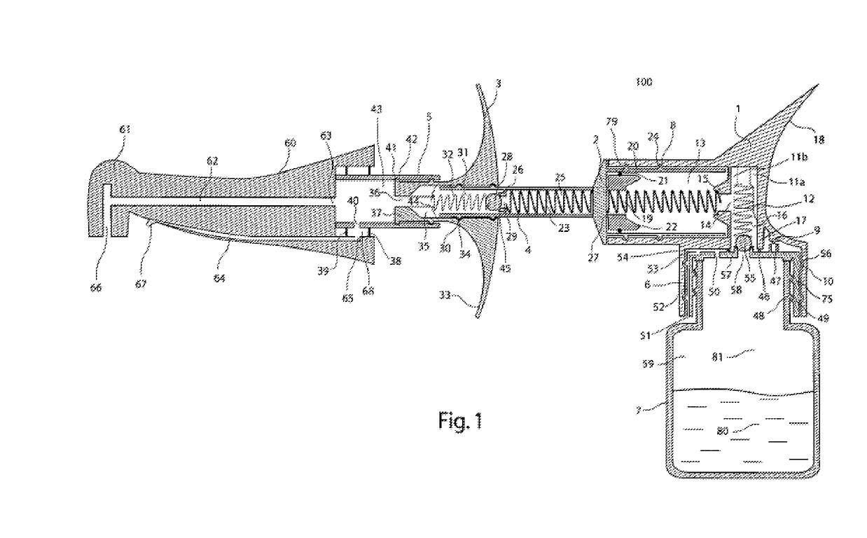

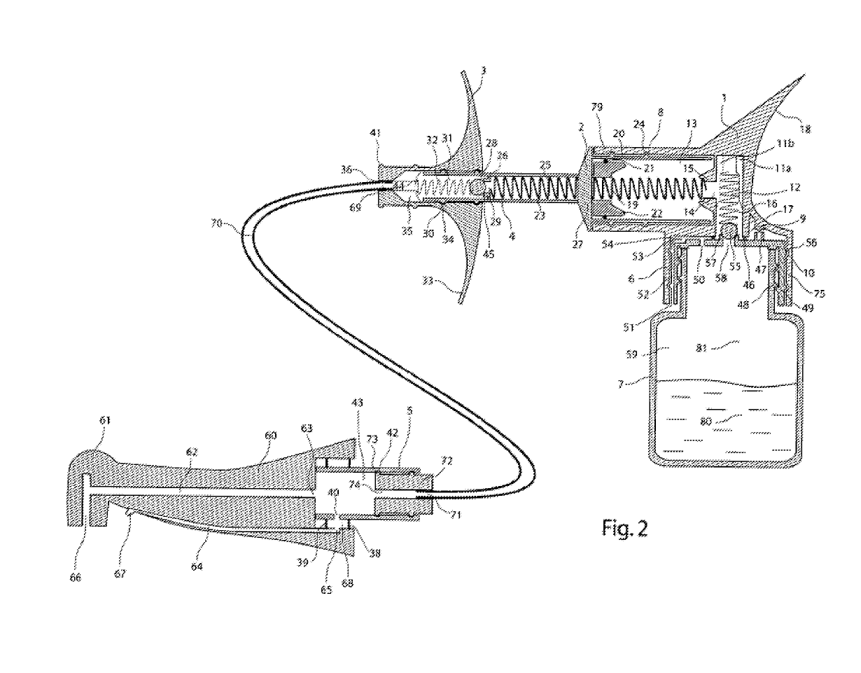

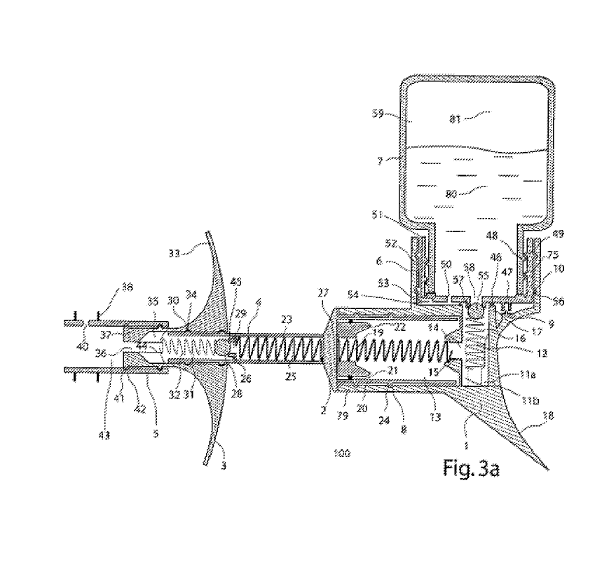

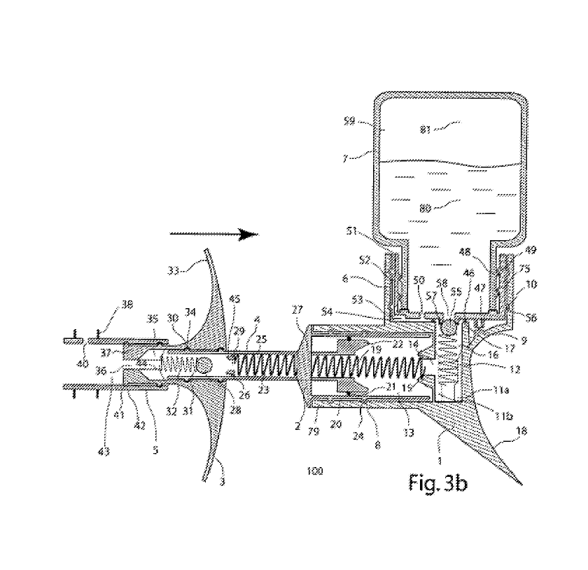

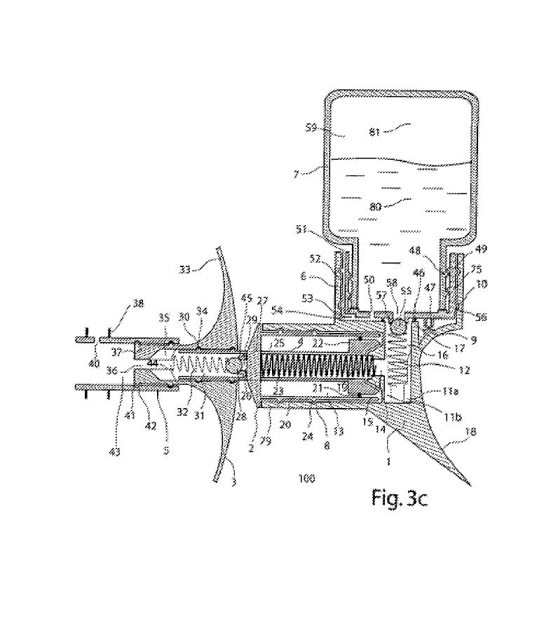

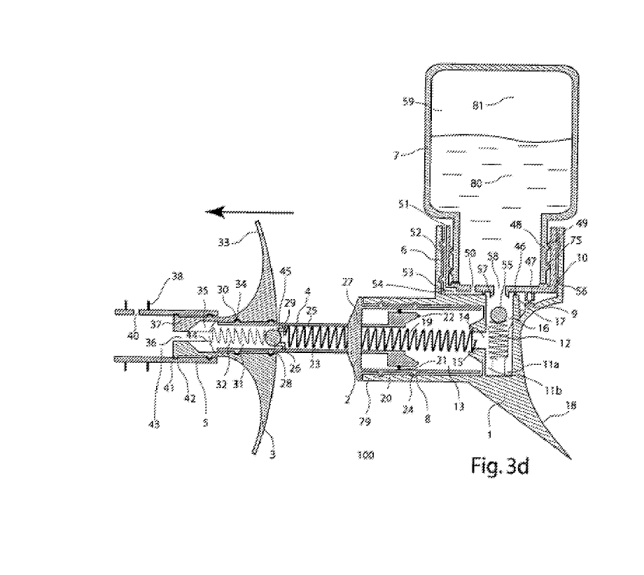

FIG. 2 shows a cross-section through a second embodiment of the inventive cleaning device 100 with a fastened container 7 and a medical instrument 60 in the second operating position. This cleaning device 100 is almost identical to the embodiment in FIG. 1. Only the piston grip 3 and the adapter 5 are connected to each other with a tube 70. The tube 70 exhibits a first tube end 69 and a second tube end 71. The first tube end 69 is placed in the discharge opening 36 of the piston grip 3. The second tube end 71 is to be so connected to the adapter 5 with the help of an intermediate element 72 that the liquid can flow out of the piston grip 3 through the tube 70 into the adapter 5. The intermediate element 72 interacts with the adapter 5 in a positive lock, whereupon the adapter 5 is connected to the intermediate element with the help of a retention groove 73, 42. In this embodiment the water beam exiting from the jet 74 impacts directly on the intake opening 63, which guarantees an optimal cleaning of the main canal 62.

The cleaning procedure is described using FIGS. 3a, 3b, 3c and 3d. FIG. 3a shows the cleaning device 100 ready to operate in the first operating position. In this position the intake valve spring 12 presses on the ball of the intake valve 55 and blocks the opening 58 of the intake valve. The discharge valve filter 32 presses on the ball of the discharge valve 28 and blocks the opening 29 of the discharge valve. Thus no liquid flows through the cleaning device.

FIG. 3b shows the cleaning device 100 in the first operating position in the dispensing mode. The hollow piston 4 is thereby to be inserted into the cylinder 2. To do so, the user presses with his finger on the anatomical curvature 33 of the piston grip 3. In the process pressure is built up in the piston area 13. The sealing ring 21 prevents the fluid from being able to exit from the piston area 13 next to the hollow piston head 22. The pressurized fluid and the intake valve spring 12, also under pressure, press the ball of the intake valve 55 against the opening 58 and in this manner close the intake valve. The sealing ring 46 prevents the fluid from exiting out of the intake valve area 16 into the area of the air intake system 54. Thus the fluid is pressed though the piston area 13 and the intake valve area 16 through the intake opening of the hollow piston 19. The fluid flows through the interior of the hollow piston 25 and exerts pressure on the ball of the discharge valve 28. This pressure is greater than the pressure of the discharge valve spring 32. As a result the ball of the discharge valve 28 is pushed away from the opening of the discharge valve 29, which causes an opening of the discharge valve 29, whereupon the fluid can flow through the discharge valve area 31, the discharge opening 36, and the area of the first fastening module 43 into the cavities of the medical instrument. Depending on the type fluid used, a different effect is attained. Depending on the type of fastening, the main cavity 62 or the secondary cavity 65 or both are filled with liquid.

FIG. 3c shows the cleaning device 100 fully emptied in the first operating position. In this position the intake valve filter 12 presses the ball of the intake valve 55 and blocks the opening 58 of the intake valve. The discharge valve filter 32 presses the ball of the discharge valve 28 and blocks the discharge valve 28 opening 29. No fluid flows through the cleaning device 100.

FIG. 3d shows the cleaning device 100 in the first operating position in the refill mode, wherein the hollow piston 4 has already been pulled 2/3 of the way out of the cylinder 2. The hollow piston 4 is pressed out of the cylinder 2 under the pressure of the hollow piston spring 23. In this manner a partial vacuum is built up in the piston area 13. The sealing ring prevents air from flowing into the piston area 13 in addition to the hollow piston head 22. The ball of the discharge valve 28 is pressed against the opening 29 of the discharge valve under a partial vacuum in the piston area 3 and in the interior of the hollow piston 25, as well as under the pressure of the discharge valve filter 32 and closes it. Thus no fluid can be suctioned out of the instrument 60. The partial vacuum on the ball of the intake valve 55 is greater than the pressure from the intake valve spring 12. Thus the ball of the discharge valve 12 is pushed away from the opening of the discharge valve 58, whereupon the opening of the discharge valve 58 is opened. The fluid flows under a partial vacuum through the intake valve area 16 out of the container 7 and through the intake opening of the piston area 14 into the piston area 13 and fills it. When the hollow piston head 22 has reached the end of the cylinder 2, the cleaning device is again ready to operate and the procedure can be continued as shown in FIG. 3a.

Repeated activation of the cleaning device 100 in the first operating position in the sequence shown in FIGS. 3a to 3d causes repeated pumping of liquid 80 out of the container 7 into the cavity 62, 64 of the medical instrument 60.

In the second operating position the steps are exactly the same with the difference that air is moved through the medical instrument by means of the cleaning device and in this manner the cavities of the medical instrument are freed of liquid deposits.

In the event the cleaning device is swiveled after a discharge of liquid approximately 180° in the direction of gravity from the first to the second operating position, the cleaning device is again filled with liquid. Only after completion of the analogous steps shown in FIGS. 3a to 3d is the cleaning device filled with air. Multiple repetitions of steps analogous to FIGS. 3a to 3d are necessary to remove the liquid from the instrument cavity.

Although the invention was described by the depiction of specific embodiments, it is apparent that numerous other embodiment variants can be created with the knowledge of this invention, for example the features of the exemplary embodiments can be combined with each other and/or individual functional units of these embodiment variants can be exchanged.

### LIST OF REFERENCE NUMBERS

1 Cylinder housing 2 Cylinder

3 Piston grip

4 Hollow piston

5 Adapter or first fastening module

6 Second fastening module

7 Container

8 Locking indentation for cylinder (2)

9 Positioning projection

10 Retention groove of the second fastening module (6)

11a Support plate for intake valve spring (12)

11b Support plate for intake valve spring (12), shown as contour

12 Intake valve spring

13 Cylinder area

14 Intake opening of piston area

15 First support platform for hollow piston spring

16 Intake valve area

17 Intake valve cylinder

18 Anatomical curvature of the cylinder housing

19 Intake opening of hollow piston (4)

20 Cylinder wall

21 Third sealing ring

22 Hollow piston head

23 Hollow piston spring

24 Locking protrusion for cylinder (2)

25 Hollow piston interior area

26 Second support platform for hollow piston spring

28 Piston opening

28 Discharge valve ball

29 Discharge valve opening

30 Locking protrusion for piston grip (3)

31 Discharge valve area

32 Discharge valve spring

33 Anatomical curvature of the piston grip (3)

34 Locking indentation

36 Discharge opening

37 Jet

38 First sealing ring

39 Second sealing ring

40 Side opening

41 Locking protrusion for adapter (5)

42 Locking indentation for jet (37)

43 Adapter space, area of the first fastening module

44 Discharge valve spring support plate

45 Support platform for the ball of the discharge valve (28)

46 Fourth sealing ring

47 Positioning indentation

48 Cylinder threading

49 Threaded insert

50 Discharge opening of air intake system

51 Intake opening of the air intake system

52 Air canal

53 Air canal section

54 Air intake system area

55 Intake valve ball

56 Locking protrusion for second fastening module (6)

57 Support platform for the ball of the intake valve (55)

58 Opening for liquid passage between container and device

59 Container cavity

60 Medical instrument

61 Tool head

62 Instrument cavity (main canal)

63 Intake opening of the main canal

64 Secondary canal

65 Intake opening of the secondary canal

66 Discharge opening of the main canal

67 Discharge opening of the secondary canal

68 Interior area between first and second sealing ring (38, 39)

69 First tube end

70 Tube

71 Second tube end

72 Intermediate element

73 Retention groove

74 Jet

75 Threaded insert

79 Dosing pump

80 Liquid

81 Air

100 Cleaning device

1. Device for cleaning medical instruments with at least one instrument cavity and at least two openings, wherein the device features a first fastening module for detachably fastening the medical instrument and a second fastening module for detachably fastening a first container for receiving liquid and/or air, wherein the device thereby exhibits a cleaning device cavity, which is so designed that with a fastened medical instrument and a fastened container, the liquid and/or air is flushed from the interior of the container through the cleaning device cavity into the instrument cavity, wherein the device features a first and a second operating position, wherein in the first operating position the liquid is flushed from the container through the cleaning device cavity into the instrument cavity and in the second operating position ambient air is flushed through the container and the cleaning device cavity into the instrument cavity.

2. Device according to claim 1, wherein by swiveling the device by approximately 180° in the direction of gravity there is a change of the operating position from the first to the second, wherein liquid is flushed in the first operating position through the second fastening module into the cleaning device cavity because of gravity.

3. Device according to claim 1, wherein the device features a dosing pump, wherein by means of the dosing pump liquid is flushed in the first operating position out of the container through the cleaning device cavity into the instrument cavity and by means of the dosing pump ambient air is flushed in the second. operating position through the container and the cleaning device cavity into the instrument cavity.

4. Device according to claim 1, wherein a second receiving element can be fastened on the second fastening module and the second receiving element features a selection module, which enables the selective removal of liquid and/or gas from the first or second receiving element.

5. Device according to claim 4, wherein the selection module features a rotatable disc with at least one opening, wherein the opening is so designed that the liquid and/or the gas can be selectively removed from the first or the second receiving element.

6. Device according to claim 1, wherein the first fastening module features a mechanism to rotate the movable parts of the medical instrument.

7. Device according to claim 1, wherein the first fastening module features a Luer-lock connection.

8. Device according to claim 1, wherein the first fastening module is an adapter.

9. Device according to claim 1, wherein the second fastening module features means to compensate the pressure in the interior of the container.

10. Method for cleaning a medical instrument by using a device according to claim 1.

11. Method according to claim 10, wherein the liquid exhibits a greater density than the atmospheric air and in the first operating position the liquid is located before the second fastening module and in the second operating position the atmospheric air is located before the second fastening module.

12. Method according to claim 10, wherein the liquid features several components and these components form a non-homogeneous mixture.

13. Method according to claim 10, wherein one of the components is oil and the volume portion of oil in the liquid is greater than 0.2% and less than 10%.

14. Method according to claim 10, wherein the volume portion of the oil in the liquid is greater than 0.3% and smaller than 0.7%.

15. Method according to claim 10, wherein one of the components consists of short-chain alcohols and the volume portion of the short-chain alcohols in the liquid is greater than 40% and less than 80%.

16. Method according to claim 10, wherein the liquid features phosphoric acid and the volume portion of the phosphoric acid in the liquid is greater than 0.1% and less than 2%.

17. Device according to claim 2, wherein the device features a dosing pump, wherein by means of the dosing pump liquid is flushed in the first operating position out of the container through the cleaning device cavity into the instrument cavity and by means of the dosing pump ambient air is flushed in the second.

18. Device according to claim 2, wherein a second receiving element can be fastened on the second fastening module and the second receiving element features a selection module, which enables the selective removal of liquid and/or gas from the first or second receiving element.

19. Device according to claim 3, wherein a second receiving element can be fastened on the second fastening module and the second receiving element features a selection module, which enables the selective removal of liquid and/or gas from the first or second receiving element.

20. Device according to claim 2, wherein the first fastening module features a mechanism to rotate the movable parts of the medical instrument.
